(19) United States
(12) Patent Application Publication
(10) Pub. No.: US 2005/0186229 A1
(43) Pub. Date: Aug. 25, 2005

(54) Pleasant-tasting aqueous liquid composition of prednisolone sodium phosphate

(76) Inventors: Emmett Clemente, Manchester, MA (US); Bhiku Patel, Chandler, AZ (US); Kuljit Bhatia, Chandler, AZ (US)

Correspondence Address: WELSH & KATZ, LTD, 120 S RIVERSIDE PLAZA, 22ND FLOOR, CHICAGO, IL, 60606, US

(21) Appl. No.: 10/786,435

(22) Filed: Feb. 24, 2004

(51) Int. Cl. A61K 31/573; A61K 9/00; A61K 31/66

(52) U.S. Cl. 424/400; 514/179; 514/125

(57) ABSTRACT

A liquid pharmaceutical composition is contemplated that comprises a pharmaceutically effective amount of prednisolone sodium phosphate (PSP) dissolved or dispersed in an aqueous medium that is free of ethanol. The aqueous medium consists essentially of water, about 3 to about 10 weight percent polyvinylpyrrolidone, about 60 to about 75 weight percent of a C₃-C₆ polyol that includes more than 55 weight percent non-reducing disaccharide or trisaccharide such as sucrose, about 0.01 to about 0.5 weight percent ammonium glycyrrhizinate and one or more flavorants, and preferably includes one or more preservatives. The liquid composition is transparent and has a pleasant taste.

### TECHNICAL FIELD

This present invention relates to a liquid drug composition, and more particularly to a room temperature-stable, microbially-protected, pleasant-tasting aqueous liquid pharmaceutical composition of prednisolone sodium phosphate.

### BACKGROUND ART

Many useful, effective drugs have a bitter taste when dissolved in liquid form or even when administered as pills or tablets. Exemplary of such drugs are acetaminophen, terfenadine, guaifenesin, trimethoprim, prednisolone, ibuprofen, prednisolone sodium phosphate, methacholine, neostigmine, epinephrine, albuterol, pseudoephedrine hydrochloride, diphenhydramine, chlorpheniramine maleate, phenothiazine, chlorpromazine, chlordiazepoxide, amitriptyline, barbiturates, diphenylhydantoin, caffeine, morphine, demerol, codeine, lomotil, lidocaine, salicylic acid, sulfonamides, prednisolone sodium phosphate, chloroquine, vitamin preparations, minerals and penicillins. These and other bitter-tasting drugs are consequently usually formatted for oral administration as coated pills or tablets or as a powder or prills within a capsule so that the bitter-tasting medicament does not contact the tongue during oral administration.

Although provision in an above coated tablet or pill form or within a capsule overcomes the problem of offensive taste for several valuable medicaments for most of the adult population that uses those drugs, many adults and many children have difficulty swallowing the pills or tablets or cannot swallow them, and thereby do not benefit from those drugs. Recently issued U.S. Pat. No. 5,455,049 illustrates one technique that was successful in overcoming the bitter taste problem associated with orally administered terfenadine.

More recently issued U.S. Pat. No. 5,763,449 and U.S. Pat. No. 5,962,461 describe a pleasant tasting-aqueous liquid composition that can contain prednisolone sodium phosphate (PSP) as the active ingredient. Work with a PSP formulation prepared in accordance with the above-noted patent provided a commercially useful pharmaceutical composition. One area for improvement of that composition lies in the fact that that composition must be kept refrigerated prior to use to maintain stability of the PSP and to protect the composition from microbial attack.

The disclosure that follows illustrates specific solution to the problems of bitter taste and oral administration of PSP that is applicable to adults and children that have difficulty swallowing or cannot swallow pills, capsules and the like, via an aqueous composition of the medication that can be stored at ambient room temperature while providing stability to the active PSP ingredient and minimizing microbial attack at an acceptable level.

### BRIEF SUMMARY OF THE INVENTION

A transparent liquid pharmaceutical composition is contemplated by the present invention. That composition contemplates a liquid pharmaceutical composition comprising a pharmaceutically effective amount of prednisolone sodium phosphate (PSP) dissolved or dispersed in an aqueous medium that is free of ethanol. The aqueous medium consists essentially of water, about 3 to about 10 weight percent polyvinylpyrrolidone (PVP), about 60 to about 75 weight percent of a C₃-C₆ polyol that includes at least 55 weight percent of a non-reducing disaccharide or trisaccharide such as sucrose, about 0.01 to about 0.5 weight percent ammonium glycyrrhizinate and one or more flavorants. That liquid composition is transparent and has a pleasant taste when orally administered; i.e., it is substantially free from having a bitter taste that would otherwise be associated with the bitter-tasting PSP. A contemplated composition is designed for storage at ambient room temperature; i.e., at about 20-35° C.

In preferred practice, the drug is present in an amount of less than about 0.5 weight percent. It is preferred that the C₃-C₆ polyol be present as a mixture of C₃ polyol and C₆ polyol, that the mixture of C₃ polyol and C₆ polyol be present in an amount of about 63 to about 70 weight percent, and that the weight ratio of the C₃ polyol to the C₆ polyol be about 1:6 to about 1:15. The PVP is preferably present at about 3 to about 7 percent.

The present invention has several benefits and advantages.

One benefit is that a contemplated composition has a pleasant taste that permits it to be administered to children without the usually observed reluctance of children to take the bitter-tasting drug.

An advantage of the invention is that a contemplated composition can be stored at ambient room temperature without fear of degradation of the prednisolone sodium phosphate active ingredient nor fear of microbial contamination.

Another benefit of the invention is that a contemplated composition can be free of ethanol so that it can be taken by children to whom an ethanol-containing pharmaceutical composition would normally not be given.

Another advantage of the invention is that a contemplated composition is transparent, homogeneously dispersed and non-settling so that one need not resuspend the medication within the composition prior to each administration and each dose contains a desired amount of the medicament.

Still further benefits and advantages of the invention will be apparent to those skilled in the art from the disclosure that follows.

### DETAILED DESCRIPTION OF THE INVENTION

The present invention contemplates a liquid pharmaceutical composition that contains prednisolone sodium phosphate (PSP), a bitter-tasting drug, as active ingredient. A contemplated composition nonetheless has at least a pleasant taste if not a good taste when administered orally.

The PSP is dissolved or dispersed in an aqueous medium that is transparent. That is, the composition of drug and ingredients other than the flavorant, even if not forming a true solution, is not cloudy or milky in the aqueous medium. It is presently not known if the aqueous medium containing the drug and other ingredients is a true solution or a non-settling dispersion, but that composition containing its various constituents discussed hereinafter is transparent as would be a true solution or a colloidal dispersion.

A contemplated pharmaceutical composition is free of ethanol (ethyl alcohol). Ethanol is often used in aqueous medicinal compositions as a solvent for the active medicament. However, because of its potential effects upon children, ethanol is not utilized in a preferred composition, or if used is present in an amount of about ten percent by volume or less.

A contemplated composition is referred to as having an aqueous medium in that water is present as a major liquid ingredient. Water is a major liquid component of a contemplated composition, but water does not constitute a majority of the composition.

A pharmaceutically effective amount of sodium prednisolone phosphate (PSP) is also present in a contemplated composition as the active ingredient. A pharmaceutically effective amount of PSP is a concentration of the drug, which when present in a predetermined volume of the composition, provides a therapeutic dosage. A contemplated amount of PSP can differ where compositions formulated for children and adults are contemplated, as well as when different amount of composition are contemplated for administration.

Therapeutic dosages of PSP are well-known and are available from the above-noted texts as well as from the Physicians' Desk Reference, Medical Economics Company, Inc., Oradell, N.J. or Goodman and Gilman's The Pharmacological Basis of Therapeutics, 8th ed., Gilman et al. eds., McGraw Hill, Inc., New York, N.Y. (1993).

Exemplary amounts of active PSP are present at about 0.1 to about 2 weight percent. Preferably, PSP is present in an amount of less than about 0.5 weight percent of the completed composition.

The determination of a bitter taste is carried out by standard, well-known practices, and is a characteristic often listed along with a description of the drug in texts such as The Merck Index, 11th ed., S. Budavari et al. eds., Merck & Co., Inc., Rahway, N.J. (1989) and Remington's Pharmaceutical Sciences, 18th ed., A. Gennaro ed., Mack Publishing Co., Easton, Pa. (1990).

A contemplated composition also contains about 60 to about 75 and more preferably about 63 to about 70 weight percent (as solids or non-volatile liquids) of a C₃-C₆ polyol. Of those polyols, at least 55 weight percent, and preferably about 60 percent by weight, of the total pharmaceutical composition is a non-reducing disaccharide or trisaccharide such as sucrose, trehalose, or raffinose, with sucrose being preferred. A non-reducing disaccharide or trisaccharide (sugar) does not reduce Fehling solution. It is noted that each trehalose, raffinose and sucrose molecule is composed of a plurality of C₆ polyols linked together. Such molecules are each deemed herein to be a C₆ polyol.

No other single polyol, or mixture of polyols, has been found that can replace a non-reducing sugar such as sucrose in the recited amount in a contemplated composition. Sucrose, for example, in the recited amount provides a benefit to both taste and protection of PSP from degradation. Indeed, fructose, the C₃-C₆ polyol preferred in a PSP composition of U.S. Pat. No. 5,763,449 and U.S. Pat. No. 5,962,461, caused a composition stored at room temperature to become more acidic on aging, with concomitant decomposition of PSP.

It is believed that the substantially higher concentration of total C₃-C₆ polyol and minimal amount of non-reducing disaccharide or trisaccharide such as sucrose required herein compared to the total C₃-C₆ polyol contemplated in a PSP composition of U.S. Pat. No. 5,763,449 or U.S. Pat. No. 5,962,461 are a major contributor to the solution of problems related to PSP stability and taste. The minimal amount of non-reducing sugar such as sucrose required herein equals the maximum amount of C₃-C₆ polyol permitted in either above patent.

Exemplary other C₃-C₆ polyols that can be present in a contemplated composition include propylene glycol, glycerin (glycerol), threose, threitol, erythrose, erythritol, ribose, arabinose, lyxose, maltitol, sorbitol, sorbose, glucose, mannose, galactose, xylose, fructose and the like. However, the presence of one or more reducing sugars such as fructose, maltose or galactose is not preferred as those polyols oxidize more readily than do non-reducing sugars, and that oxidation is believed to lead to acidification of the composition and decomposition of the PSP. Maltitol is another molecule containing to linked C₆ polyols that is deemed therefore to be a C₆ polyol. A C₃-C₆ polyol serves the dual function of being a solvent for the system as well as being a bitter flavor masking agent.

In one preferred embodiment, a mixture of two or more C₃-C₆ polyols is utilized. Such a preferred mixture utilizes a C₃ polyol such as glycerin or 1,3-propanediol (propylene glycol), with both of those C₃ polyols being preferred for use.

One or more C₆ reducing sugar oligosaccharide polyols such as sorbitol USP 70%, maltitol NF at 75 percent solids, liquid fructose (an α-hydroxyketose that is a reducing sugar) such as that available under the mark KRYSTAR™, from A.E. Staley Mfg. Co., Decatur, Ill. that is sold as an aqueous liquid about 77 weight percent of which is fructose, as well as sugars such as glucose, xylitol and the like can also be present.

This C₃-C₆ polyol mixture, when utilized, is typically present at a ratio weight of 1:6 to about 1:15, C₃ to C₆ polyol, as non-volatiles. In another preferred embodiment, polyols other than a C₆ polyol constitute less than about 5 weight percent of the total composition.

In addition to the water, PSP and C₃-C₆ polyol, a contemplated composition also contains about 3 to about 10 weight percent polyvinylpyrrolidone (PVP), and more preferably about 3 to about 7 weight percent PVP. PVP is commercially available from a number of suppliers under a number of designations. The PVPs sold under the Trademark KOLLIDON® K25, K30 and K90 having weight-average molecular weights of 28,000-34,000, 44,000-54,000 and 1,000,000-1,500,000, respectively, are preferred for use here, with the K25 and K30 being most preferred.

PVP is dissolved or dispersed in the water of the aqueous medium and serves to assist in dissolving or dispersing the bitter-tasting drug in that medium, as well as masking the flavor of the bitter-tasting drug. The disclosures of Volker Bühler's book, Kollidon, BASF Aktiengesellshaft, Ludwigshafen, Germany (1992) teach the use of PVP as both a solubilization aid for several drugs as well as for masking the bitter taste of acetaminophen. An exemplary formulation for an oral PVP- and acetaminophen-containing composition is provided at page 113, Table 81 of the above Bühler text.

Surprisingly, the use of the above C₃-C₆ polyols together with the PVP is not sufficient to suitably mask the bitter taste of the bitter-tasting PSP. This fact remains even when further sweeteners such as sodium saccharin USP present at 0.05-2 weight percent or aspartame present at about 0.1 to about 2 weight percent and further flavorants are admixed with the composition. A further debittering agent is still required to be present.

That further debittering agent is found to be ammonium glycyrrhizinate that can be present at about 0.01 to about 0.5 weight percent as ammonium glycyrrhizinate itself. The ammonium glycyrrhizinate is preferably present at a weight ratio to the PSP of about 1:100 to about 1:5, more preferably about 1:50 to about 1:10, and most preferably at weight ratio of about 1:20 ammonium glycyrrhizinate to PSP.

Ammonium glycyrrhizinate is available as a 10 weight percent solution in glycerin or propylene glycol from MacAndrews & Forbes Company of Camden N.J. under the name MAGNASWEET® MM110 or MM115, and also as a white, amorphous powder as MM150. Ammonium glycyrrhizinate is the monoammonium salt of a triterpenoid saponin that consists of an aglycone of glycyrrhetic acid and a sugar moiety of two glucuronic acid units linked to each other. This material is said by its manufacturer to be about 50 to about 100 times sweeter than sucrose, and is known to be useful in masking bitterness.

Although ammonium glycyrrhizinate is a known bitterness-masking agent as is PVP, neither material alone or with the before-discussed sweeteners and flavorants is sufficient to mask the bitter taste of a contemplated bitter-tasting drug. Rather, PVP and ammonium glycyrrhizinate appear to potentiate each other to provide the desired bitterness-masking effect.

The mechanism by which the bitterness-masking is achieved is unknown. However, without wishing to be bound by theory, it is believed that a complex is formed among the PVP, drug and ammonium glycyrrhizinate, particularly because so little of the glycyrrhizinate is present.

The before-mentioned Bühler, Kollidon, BASF Aktiengsellshaft, Ludwigshafen, Germany (1992) book teaches that PVP forms complexes with aromatic compounds, particularly those drugs also having hydrophilic groups that can form hydrogen bonds such as carboxyl, hydroxyl and amine groups. See also, Horn et al., J. Pharm. Sci., 71:1021-126 (1982). The contemplated bitter-tasting drugs have one or more rings, most of which are aromatic, and so it is thought that PVP forms a complex with the bitter-tasting drug. Table 20 at page 40 of Bühler's book lists interaction constants for several such complexes, although no such interaction constant could be determined for trimethoprim, which is quite useful here. See also, Horn et al., J. Pharm. Sci., 71:1021-1026 (1982).

Ammonium glycyrrhizinate contains no aromaticity, but has several hydrophilic groups such as hydroxyls and carboxyl groups and a hydrophobic aglycone portion that can be solvated by the PVP polymeric backbone. It is consequently believed that the three components form a presently undefined complex in the aqueous medium, and that that complex acts to shield taste buds from the bitterness inherently present in the bitter-tasting drug.

A contemplated composition has a final pH value of about 6 to about 8, and preferably about 6.5 to about 7.5, and more preferably about 6.7 to about 7.4, and most preferably about 6.8 to about 7.2. Sodium hydroxide (1 N) and hydrochloric acid (10 N) or citric acid and sodium citrate are typically used for pH value adjustments and maintenance. Mono- and dibasic sodium or potassium phosphate salts and other salts can also be used as a buffer system to maintain the pH value of a contemplated composition. It is preferred to include a minimal amount, e.g., less than about 5 percent, and more preferably less than about 2 percent, of acid and base as buffering salts such as the above mono- and dibasic phosphate salts, because too much salt present in the composition can result in an unpleasant taste being imparted to the composition. Such high concentrations are typically not needed because of the preferred absence of reducing sugars that produce acid groups upon oxidation.

As was noted previously, a contemplated composition can also contain additional sweeteners, and flavorants, as well as colorants and thickeners. Flavorants such as bubble gum and chocolate flavors can provide opacity or translucency to a contemplated composition, while the composition other than the flavorant is transparent. Exemplary thickeners include sodium alginate, gelatin or a polyalkylene oxide such as the polyoxyethylene-polyoxypropylene-polyethylene terpolymer available under the name PLURONIC® F68 having an average of 75 polymerized ethylene oxide units on either side of 30 polymerized propylene oxide units, F-87 having 62 polymerized ethylene oxide units on either side of 39 polymerized propylene oxide units, or F-88 having an average of about 97 polymerized ethylene oxide groups on either side of about 39 polymerized propylene oxide groups that are available from BASF, Mount Olive, N.J. A contemplated aqueous liquid pharmaceutical composition has a viscosity at 25 [symbol] C. between that of water and about that of corn syrup at 25 [symbol] C.

It has been found that a contemplated composition for room temperature storage is unexpectedly difficult to protect from microbial attack while maintaining the activity of the PSP. By comparison, a PSP composition of U.S. Pat. No. 5,763,449 and U.S. Pat. No. 5,962,461 contained ethanol and had to be refrigerated to provide the desired PSP stability and freedom from microbial attack.

Conventional preservatives such as sodium benzoate NF, methylparaben NF and propylparaben NF as are discussed in U.S. Pat. No. 5,763,449 and U.S. Pat. No. 5,962,461 are preferably also present herein. Inclusion of those preservatives and removal of PVP from a test composition showed that protection from microbial attack was present, indicating that PVP may be binding to and thereby partially removing one or more of those preservatives from effective use in a contemplated composition.

Those conventional preservatives, while useful, were ineffective in providing the desired room temperature freedom from microbial attack for a composition contemplated here. Indeed, Remington's Pharmaceutical Sciences, 18th ed., A. Gennaro ed., Mack Publishing Co., Easton, Pa. (1990) at page 1173 notes that sodium benzoate, used herein is not effective at a pH value greater than about 4. To the contrary, however, removal of the benzoate from a contemplated composition led to increased microbial attack, so it is concluded that that preservative is in fact useful at the higher pH values utilized herein.

It has been further been found that inclusion of sodium or potassium sorbate with the above preservatives along with the above recited amount of C₃-C₆ polyol can provide the desired room temperature stability of PSP and a desired level of freedom from microbial attack. Benzyl alcohol can also be present as a preservative. Each of the preservatives is utilized in an effective amount that is less than 1 percent of the composition, and more typically at about 0.01 to about 0.75 weight percent.

Composition Preparation

A contemplated aqueous liquid pharmaceutical composition is readily prepared. Thus, in an exemplary procedure where a C₃ polyol is utilized, a solution or dispersion of about 30 weight percent PVP is prepared in water. About one part PSP is slurried with about 5 parts by weight C₃ polyol (glycerin or propylene glycol or both). The two compositions are admixed and heated to a temperature of about 45 [symbol] C. with continued agitation. Agitation is continued at that temperature until a clear, non-settling solution or dispersion is formed, which generally takes about 30 minutes. Where less than about 5 weight percent C₃ polyol is used, as is preferred here, the PSP is admixed directly with the aqueous PVP.

The aqueous composition so formed is cooled at a temperature below about 30 [symbol] C. and the ammonium glycyrrhizinate, other C₃-C₆ polyols, flavorants, colorant if used and remaining ingredients are admixed until a homogenous composition is obtained. These additions are typically carried out serially, with admixture to homogeneity between each admixture. The preservatives are added and the pH value is thereafter adjusted as required. The examples that follow illustrate these procedures more fully.

### BEST MODE FOR CARRYING OUT THE INVENTION

### EXAMPLE 1

### COMPARATIVE EXAMPLE

### U.S. Pat. No. 5,962,461 Ethanol-Containing Prednisolone Sodium Phosphate Oral Liquid

Two pleasant-tasting liquids for oral administration containing prednisolone sodium phosphate as active ingredient were prepared containing the ingredients and amounts shown in the table below for liquid 1 and liquid 2.

| | | Liquid 1 | Liquid 2 |
|---|---|---|---|
| Ingredient and Number | | (w/v or w/w %) | (w/v or w/w %) |
| 1. | Prednisolone Sodium | 0.134 | 0.403 |
| | Phosphate USP | | |
| 2. | Polyvinylpyrrolidone, | 5.0 | 5.0 |
| | USP (PVP; K25) | | |
| 3. | Ethanol, USP | 1.71 | 1.71 |
| 4. | Purified Water, USP | 20.0 | 20.0 |
| 5. | Sodium Benzoate, NF | 0.15 | 0.15 |
| 6. | Monoammonium | 2.0 | 2.0 |
| | Glycyrrhizinate | | |
| | (10% solids)¹ | | |
| 7. | Sorbitol Solution, USP | 10.0 | 10.0 |
| | (70% solids) | | |
| 8. | Sodium Hydroxide, USP | q.s. | q.s |
| | (1 N) | | |
| 9. | Citric Acid, USP | q.s. | q.s |
| | (50%) | | |
| 10. | Maltitol Solution, NF | q.s. | 20.0 |
| | (75% solids) | | |
| 11. | Favorant | 0.65 | 0.75 |
| 12. | Liquid Fructose | — | q.s |
| | (77.0-77.5% solids) | | |

¹A 10% solids solution in glycerin or propylene glycol from MacAndrews & Forbes Co.

Purified water, USP was charged into a kettle and agitation of the water begun. The Povidone 25, USP was slowly added to the mixing water and the resulting admixture agitated until all of the Povidone 25, USP was dissolved to form phase one.

Further purified water, USP was added to a separate vessel and stirring of the water begun. To the stirring solution was added prednisolone sodium phosphate, USP, and stirring was continued until all particles were dissolved to form phase two.

Still further purified water, USP was added to a third vessel and mixing of the water began. Sodium benzoate, NF was admixed with agitation until all particles were dissolved to form phase three.

Phase one, phase two and phase three were admixed together with agitation, followed by admixture of the Sorbitol Solution 70%, USP, the Magnasweet™, Ethanol, Flavorant, Maltitol solution, NF, and the resulting composition diluted qs with liquid fructose. Agitation was continued to homogeneity. The pH value was measured and adjusted to 7.0 (±0.3) with citric acid or sodium hydroxide as necessary.

After storage of above Liquid 2 at a temperature of 40° C. and 75% relative humidity (RH) for a time period of three months, the composition changed from a clear yellow color to a clear yellow-orange and then a clear amber color, the pH value dropped from 6.9 to 5.4 and the percentage of prednisolone sodium phosphate dropped from 102 percent to 83 percent. Similar samples stored at 30° C. and 60% RH or at 25° C. and 60% RH exhibited no color changes, a drop of less than one pH unit and a 6 percent or no loss of prednisolone sodium phosphate over the same time period. Those results indicated a temperature-dependent acidification that led to decomposition of the prednisolone sodium phosphate.

### EXAMPLE 2

### Room Temperature Storage Stable Aqueous Composition of Sodium Prednisolone Phosphate

An aqueous, room temperature storage stable PSP composition containing the following amounts of ingredients were prepared and subjected to 25° C. and 60% RH storage for a period of nine months with very good product stability. The composition was also placed under accelerated storage conditions at a temperature of 40° C. and 75% RH for a period of three months, a usually strong indication of two years of shelf life at room temperature.

Room Temperature Storage Stable PSP Composition

| | | Liquid |
|---|---|---|
| Ingredient and Number | | (w/v or w/w %) |
| 1. | Prednisolone Sodium | 0.4032 |
| | Phosphate, USP | |
| 2. | Sucrose NF | 60 |
| 3. | Polyvinylpyrrolidone, | 5 |
| | USP (PVP; K25) | |
| 4. | Propylene Glycol, USP | 1 |
| 5. | Sorbitol Solution, | 5 |
| | (70% solids), USP | |
| 6. | Monoammonium Glycyrrhizinate | 2.0 |
| | (10% solids), Magnasweet ™ | |
| 7. | Favorant (Grape) | 0.75 |
| 8. | Sodium Benzoate, NF | 0.15 |
| 9. | Methylparaben, NF | 0.1 |
| 10. | Propylparaben, NF | 0.05 |
| 11. | Potassium Sorbate, NF | 0.5 |
| 12. | Edetate Disodium | 0.05 |
| | Dihydrate, USP | |
| 13. | Sodium Hydroxide, USP (1 N) | q.s., pH 7.0 |
| 14. | Hydrochloric Acid, USP (1 N) | q.s., pH 7.0 |
| 15. | Dibasic Sodium Phosphate, | 1.45 |
| | Anhydrous, USP | |
| 16. | Monobasic Sodium Phosphate, | 0.16 |
| | Monohydrate, USP | |
| 17. | Purified Water, USP | qs (about 23.39) |

A 1000 liter contemplated aqueous composition of PSP was prepared by the admixture of three substantially homogeneous sub-compositions. The first sub-composition was prepared by admixing the propylene glycol (4), methylparaben (9), propylparaben (10) and grape flavor (7) until substantially homogeneous. The second sub-composition was prepared from a first portion of purified water (17), about 37 percent of the total compostion's weight, was heated to about 65° C.±about 5° C. and then admixed with the sucrose (2), soribitol (5) Magnasweet™ 110 until substantially homogeneous, and the resulting admixture was cooled to a temperature of about 40° C. or below, at which time the povidone was admixed with stirring to substantial homogeneity. The third sub-composition was prepared by admixture and agitation to substantial homogeneity of a second portion of purified water that constituted about 15 percent of the total composition with the mono- and dibasic sodium phosphates (16 and 15), EDTA (12), sodium benzoate (8) prednisolone sodium phosphate (1) and potassium sorbate (11).

The second and third sub-compositions were admixed to substantial homogeneity, followed by admixture of the first sub-composition and further mixing to substantial homogeneity. The pH value was adjusted to the desired value of about 7.0, and remainder of the purified water (15), about 4 percent, was then admixed to complete the preparation of the desired composition. It is to be understood that such a desired composition can also be prepared in other manners.

Illustrative lots submitted for microbiological testing pursuant to the current USP 26 for both (a) Preservative Effectiveness Test (PET), Chapter <51>, and (b) Microbial Limits, Chapter <61>, as well as for analytical Assay Determination under the current USP 26, Validation Compendia, Chapter <1225>, all passed their respective testing.

Each of the patents and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

1. A liquid pharmaceutical composition comprising a pharmaceutically effective amount of prednisolone sodium phosphate (PSP) dissolved or dispersed in an aqueous medium that is free of ethanol, said aqueous medium consisting essentially of water, about 3 to about 10 weight percent polyvinylpyrrolidone, about 60 to about 75 weight percent of a C₃-C₆ polyol and including more than 55 weight percent of a non-reducing disaccharide or trisaccharide, about 0.01 to about 0.5 weight percent ammonium glycyrrhizinate and one or more flavorants, said liquid composition being transparent and having a pleasant taste.

2. The liquid pharmaceutical composition according to claim 1 wherein said polyvinylpyrrolidone is present at about 3 to about 7 weight percent.

3. The liquid pharmaceutical composition according to claim 1 wherein said C₃-C₆ polyol is present as a mixture of C₃ polyol and C₆ polyol.

4. The liquid pharmaceutical composition according to claim 3 wherein said mixture of C₃ polyol and C₆ polyol is present in an amount of about 63 to bout 70 weight percent.

5. The liquid pharmaceutical composition according to claim 1 wherein the weight ratio of said C₃ polyol to said C₆ polyol is about 1:6 to about 1:15.

6. The liquid pharmaceutical composition according to claim 1 wherein the non-reducing disaccharide or trisaccharide is sucrose.

7. A liquid pharmaceutical composition comprising a pharmaceutically effective amount of prednisolone sodium phosphate (PSP) dissolved or dispersed in an aqueous medium that is free of ethanol, said aqueous medium consisting essentially of water, about 3 to about 10 weight percent polyvinylpyrrolidone, a mixture of C₃ polyol and C₆ polyol is present in an amount of about 63 to about 70 weight percent in which the weight ratio of said C₃ polyol to said C₆ polyol is about 1:6 to about 1:15 and said mixture includes more than 55 weight percent sucrose, about 0.01 to about 0.5 weight percent ammonium glycyrrhizinate and one or more flavorants, said liquid composition being transparent and having a pleasant taste.

8. The liquid pharmaceutical composition according to claim 7 wherein said PSP is present in an amount of less than about 0.5 weight percent.

9. The liquid pharmaceutical composition according to claim 7 that further includes dissolved buffer salts that provide a buffered pH value of about 6 to about 8.

10. The liquid pharmaceutical composition according to claim 7 wherein said C₃ polyol is itself a mixture of glycerol and propylene glycol.

11. The liquid pharmaceutical composition according to claim 7 that further includes a preservative effective amount of one or both of (i) sodium or potassium sorbate and (ii) benzyl alcohol.

12. A liquid pharmaceutical composition comprising a pharmaceutically effective amount of prednisolone sodium phosphate present in an amount of less than about 0.5 weight percent dissolved or dispersed in an aqueous medium that is free of ethanol and buffered at a pH value of about 6 to about 8, said aqueous medium consisting essentially of water, buffer salts, about 3 to about 10 weight percent polyvinylpyrrolidone, a mixture of C₃ polyol and C₆ polyol is present in an amount of about 63 to about 70 weight percent in which the weight ratio of said C₃ polyol to said C₆ polyol is about 1:6 to about 1:15, said C₃ polyol is comprised of a mixture of glycerol and propylene glycol and said mixture of C₃ polyol and C₆ polyol includes more than 55 weight percent sucrose, about 0.01 to about 0.5 weight percent ammonium glycyrrhizinate and one or more flavorants, said liquid composition being transparent and having a pleasant taste.

13. The liquid pharmaceutical composition according to claim 12 that further includes one or both of (i) sodium or potassium sorbate and (ii) benzyl alcohol present at less than 1 percent of the composition.

14. The liquid pharmaceutical composition according to claim 13 wherein said one or both of (i) sodium or potassium sorbate and (ii) benzyl alcohol is present in an amount of about 0.01 to about 0.75 weight percent.
